(19) United States
(12) Patent Application Publication
(10) Pub. No.: US 2003/0017554 A1
(43) Pub. Date: Jan. 23, 2003

(54) Process for the fermentative preparation of L-amino acids using strains of the enterobacteriaceae family

(76) Inventors: Mechthild Rieping, Bielefeld (DE); Georg Thierbach, Bielefeld (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/076,416

(22) Filed: Feb. 19, 2002

Related U.S. Application Data
(63) Continuation of application No. 09/987,541, filed on Nov. 15, 2001, now abandoned.
(60) Provisional application No. 60/283,612, filed on Apr. 16, 2001.
(60) Provisional application No. 60/248,210, filed on Nov. 15, 2000.

(30) Foreign Application Priority Data
Mar. 14, 2001 (DE) 101 12 107.5

(51) Int. Cl. C12P 13/04; C12P 13/08; C12P 13/06; C12N 1/21

(52) U.S. Cl. 435/106; 435/115; 435/116; 435/252.3

(57) ABSTRACT

A process for the fermentative preparation of an L-amino acid which entails the steps of:

a) fermenting microorganisms of the Enterobacteriaceae family which produce the desired L-amino acid and in which at least the poxB gene or nucleotide sequences which code therefor are attenuated, in particular eliminated,

b) concentrating the L-amino acid in the medium or in the cells of the bacteria, and

c) isolating the L-amino acid.

### BACKGROUND OF THE INVENTION

1. Field of the Invention

The present invention relates to a process for the fermentative preparation of L-amino acids, in particular L-threonine, L-lysine and L-valine, using strains of the Enterobacteriaceae family in which the poxB gene is attenuated.

2. Description of the Background

L-Amino acids, in particular L-threonine, L-lysine and L-valine are used in human medicine and in the pharmaceutical and foodstuff industries and, very particularly, in animal nutrition.

It is known to prepare L-amino acids by fermenting strains of Enterobacteriaceae, in particular Escherichia coli (E. coli) and Serratia marcescens. Due to the importance of these processes, work is constantly being undertaken to improve them. Improvements can relate to fermentation measures, such as e.g. stirring and supply of oxygen, or the composition of the nutrient media, such as e.g. the sugar concentration during fermentation, or product work-up by e.g. ion exchange chromatography, or the intrinsic output properties of the microorganism itself.

Methods of mutagenesis, selection and mutant selection are currently used to improve the output properties of these microorganisms. Strains which are resistant to antimetabolites, such as the threonine analogue α-amino-β-hydroxyvaleric acid (AHV), or which are auxotrophic for metabolites of regulatory importance and produce L-amino acids, such as e.g. L-threonine, are obtained in this manner.

Recombinant DNA methodologies have also been employed for some years in improving strains of the Enterobacteriaceae family which produce L-amino acids, by amplifying individual amino acid biosynthesis genes and investigating the effect thereof on the production.

However, a need exists for an improved fermentative process for the production of L-amino acids, such as L-threonine, L-lysine and L-valine.

### SUMMARY OF THE INVENTION

Accordingly, it is an object of the present invention to provide methods for improved fermentative preparation of L-amino acids, in particular L-threonine, L-lysine and L-valine.

In particular, it is an object of the present specification to provide a process for the fermentative preparation of an L-amino acid, which entails the steps of:

a) fermenting microorganisms of the Enterobacteriaceae family which produce an L-amino acid, in which at least pox B gene or nucleotide sequences coding therefor are attenuated or eliminated;

b) concentrating the L-amino acid in the medium or in the cells of the bacteria; and

c) isolating the L-amino acid.

### BRIEF DESCRIPTION OF THE DRAWINGS

FIG. 1 illustrates replacement vector, pMAK705ΔpoxB.

FIG. 2 illustrates plasmid pMW218gdhA.

FIG. 3 illustrates plasmid pMW219rhtC.

### DETAILED DESCRIPTION OF THE PREFERRED EMBODIMENTS

The present invention provides a process for the fermentative preparation of L-amino acids, in particular L-threonine, L-lysine or L-valine, using microorganisms of the Enterobacteriaceae family which, in particular, already produce these amino acids and in which the nucleotide sequence which codes for the enzyme pyruvate oxidase (EC 1.2.2.2) (poxB gene) is attenuated.

The term “attenuation” in this connection means the reduction or even the elimination of the intracellular activity of one or more enzymes (proteins) in a microorganism which are coded by the corresponding DNA, for example by using a weak promoter or a gene or allele which codes for a corresponding enzyme with a low activity or inactivates the corresponding enzyme (protein) or gene, and optionally combining these measures.

The process entails carrying out the following steps:

a) fermenting microorganisms of the Enterobacteriaceae family in which at least the poxB gene is attenuated,

b) concentrating a produced L-amino acid in the medium or in the cells of the microorganisms of the Enterobacteriaceae family, and

c) isolation of the produced L-amino acid.

The microorganisms of the present invention produce L-amino acids from glucose, sucrose, lactose, fructose, maltose, molasses, optionally starch, optionally cellulose or from glycerol and ethanol. They are representatives of the Enterobacteriaceae family of the genera Escherichia, Erwinia, Providencia and Serratia. The genera Escherichia and Serratia are preferred. Of the genus Escherichia the species Escherichia coli, and of the genus Serratia, the species Serratia marcescens are to be mentioned in particular.

Suitable strains, which produce L-threonine, for example, of the genus Escherichia, in particular of the species Escherichia coli, are, for example:

Escherichia coli TF427

Escherichia coli H4578

Escherichia coli KY10935

Escherichia coli VNIIgenetika MG442

Escherichia coli VNIIgenetika M1

Escherichia coli VNIIgenetika 472T23

Escherichia coli BKIIM B-3996

Escherichia coli kat 13

Escherichia coli KCCM-10132

Suitable L-threonine-producing strains of the genus Serratia, in particular of the species Serratia marcescens, are, for example:

Serratia marcescens HNr21

Serratia marcescens TLr156

Serratia marcescens T2000.

Strains from the Enterobacteriaceae family which produce L-threonine preferably have, inter alia, one or more genetic or phenotypic features, such as resistance to α-amino-β-hydroxyvaleric acid, resistance to thialysine, resistance to ethionine, resistance to α-methylserine, resistance to diaminosuccinic acid, resistance to α-aminobutyric acid, resistance to borrelidin, resistance to rifampicin, resistance to valine analogues, such as, for example, valine hydroxamate, resistance to purine analogues, such as, for example, 6-dimethylaminopurine, a need for L-methionine, optionally a partial and compensatable need for L-isoleucine, a need for meso-diaminopimelic acid, auxotrophy with respect to threonine-containing dipeptides, resistance to L-threonine, resistance to L-homoserine, resistance to L-lysine, resistance to L-methionine, resistance to L-glutamic acid, resistance to L-aspartate, resistance to L-leucine, resistance to L-phenylalanine, resistance to L-serine, resistance to L-cysteine, resistance to L-valine, sensitivity to fluoropyruvate, defective threonine dehydrogenase, optionally a capacity for sucrose utilization, enhancement of the threonine operon, enhancement of homoserine dehydrogenase I-aspartate kinase I, preferably of the feedback-resistant form, enhancement of homoserine kinase, enhancement of threonine synthase, enhancement of aspartate kinase, optionally of the feedback-resistant form, enhancement of aspartate semialdehyde dehydrogenase, enhancement of phosphoenol pyruvate carboxylase, optionally of the feedback-resistant form, enhancement of phosphoenol pyruvate synthase, enhancement of transhydrogenase, enhancement of the RhtB gene product, enhancement of the RhtC gene product, enhancement of the YfiK gene product, enhancement of a pyruvate carboxylase, and attenuation of acetic acid formation.

In accordance with the present invention, it has been found that microorganisms of the Enterobacteriaceae family produce L-amino acids, for example L-threonine, in an improved manner after attenuation, in particular elimination, of the poxB gene, which codes for pyruvate oxidase (EC number 1.2.2.2).

It has furthermore been found that microorganisms of the Enterobacteriaceae family form lower concentrations of the undesirable by-product acetic acid after attenuation, in particular elimination, of the poxB gene, which codes for pyruvate oxidase (EC number 1.2.2.2).

The nucleotide sequence of the poxB gene of Escherichia coli has been published by Grabau and Cronan (Nucleic Acids Research. 14 (13), 5449-5460 (1986)) and can also be found from the genome sequence of Escherichia coli published by Blattner et al. (Science 277, 1453-1462 (1997), under Accession Number AE000188. The nucleotide sequence of the poxB gene of Escherichia coli is shown in SEQ ID No. 1 and the amino acid sequence of the associated gene product is shown in SEQ ID No. 2.

The poxB genes described in the text references mentioned can be used according to the invention. Alleles of the poxB gene which result from the degeneracy of the genetic code or due to “sense mutations” of neutral function can furthermore be used.

To achieve an attenuation, for example, expression of the poxB gene or the catalytic properties of the enzyme protein can be reduced or eliminated. The two measures can optionally be combined.

The reduction in gene expression can take place by suitable culturing, by genetic modification (mutation) of the signal structures of gene expression or also by the antisense-RNA technique. Signal structures of gene expression are, for example, repressor genes, activator genes, operators, promoters, attenuators, ribosome binding sites, the start codon and terminators. The expert can find information in this respect, inter alia, for example, in Jensen and Hammer (Biotechnology and Bioengineering 58: 191-195 (1998)), in Carrier and Keasling (Biotechnology Progress 15, 58-64 (1999), Franch and Gerdes (Current Opinion in Microbiology 3, 159-164 (2000)) and in known textbooks of genetics and molecular biology, such as, for example, the textbook of Knippers (“Molekulare Genetik [Molecular Genetics]”, 6th edition, Georg Thieme Verlag, Stuttgart, Germany, 1995) or that of Winnacker (“Gene und Klone [Genes and Clones]”, VCH Verlagsgesellschaft, Weinheim, Germany, 1990).

Mutations which lead to a change or reduction in the catalytic properties of enzyme proteins are known from the prior art. Examples which may be mentioned are the works of Qiu and Goodman (Journal of Biological Chemistry 272: 8611-8617 (1997)), Yano et al. (Proceedings of the National Academy of Sciences, USA 95, 5511-5515 (1998), Wente and Schachmann (Journal of Biological Chemistry 266, 20833-20839 (1991). Summarizing descriptions can be found in known textbooks of genetics and molecular biology, such as e.g. that by Hagemann (“Allgemeine Genetik”, Gustav Fischer Verlag, Stuttgart, 1986).

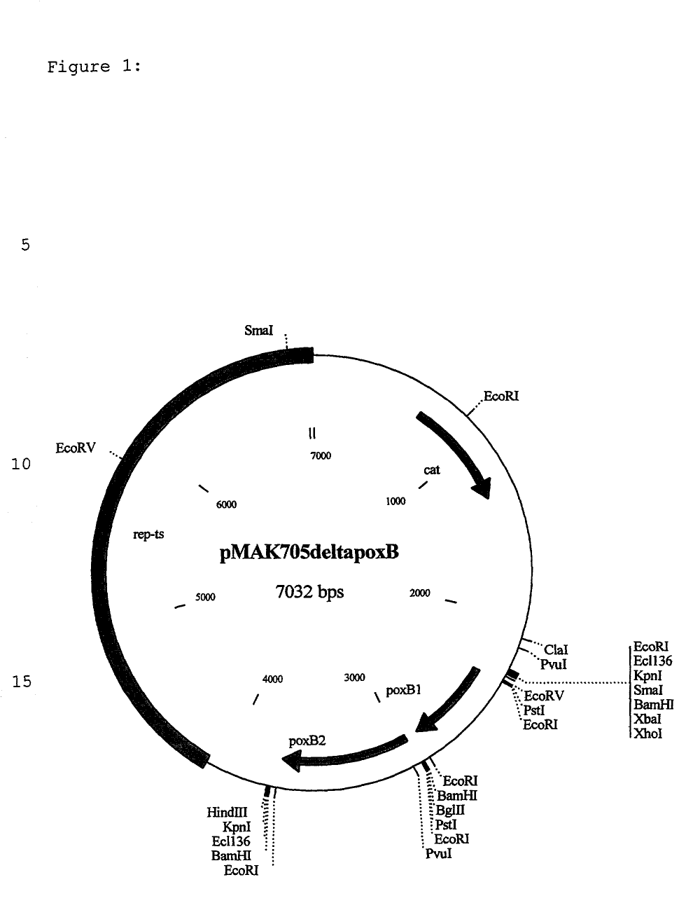

Possible mutations are transitions, transversions, insertions and deletions. Depending on the effect of the amino acid exchange on the enzyme activity, “missense mutations” or “nonsense mutations” are referred to. Insertions or deletions of at least one base pair in a gene lead to “frame shift mutations”, which lead to incorrect amino acids being incorporated or translation being interrupted prematurely. Deletions of several codons typically lead to a complete loss of the enzyme activity. Instructions on generation of such mutations are prior art and can be found in known textbooks of genetics and molecular biology, such as e.g. the textbook by Knippers (“Molekulare Genetik [Molecular Genetics]”, 6th edition, Georg Thieme Verlag, Stuttgart, Germany, 1995), that by Winnacker (“Gene und Klone ”, VCH Verlagsgesellschaft, Weinheim, Germany, 1990) or that by Hagemann (“Allgemeine Genetik ”, Gustav Fischer Verlag, Stuttgart, 1986). An example of a plasmid with the aid of which the poxB gene of Escherichia coli can be attenuated, in particular eliminated, by position-specific mutagenesis is the plasmid pMAK705ΔpoxB (FIG. 1). In addition to residues of polylinker sequences, it contains only a part of the 5′ and a part of the 3′ region of the poxB gene. A 340 bp long section of the coding region is missing (deletion). The sequence of this DNA which can be employed for mutagenesis of the poxB gene is shown in SEQ ID No. 3.

The deletion mutation of the poxB gene can be incorporated into suitable strains by gene or allele replacement.

A conventional method is the method, described by Hamilton et al. (Journal of Bacteriology 174, 4617-4622 (1989)), of gene replacement with the aid of a conditionally replicating pSC101 derivative pMAK705. Other methods described in the prior art, such as, for example, those of Martinez-Morales et al. (Journal of Bacteriology 1999, 7143-7148 (1999)) or those of Boyd et al. (Journal of Bacteriology 182, 842-847 (2000)), can likewise be used.

After replacement has taken place, the strain in question contains the form of the ΔpoxB allele shown in SEQ ID No. 4, which is also provided by the invention.

It is also possible to transfer mutations in the poxB gene or mutations which affect expression of the poxB gene into various strains by conjugation or transduction.

It may furthermore be advantageous for the production of L-amino acids, in particular L-threonine, with strains of the Enterobacteriaceae family to enhance one or more enzymes of the known threonine biosynthesis pathway or enzymes of anaplerotic metabolism or enzymes for the production of reduced nicotinamide adenine dinucleotide phosphate, in addition to the attenuation of the poxB gene.

The term “enhancement” in this connection means an increase in the intracellular activity of one or more enzymes or proteins in a microorganism which are coded by the corresponding DNA, for example by increasing the number of copies of the gene or genes, using a potent promoter or a gene which codes for a corresponding enzyme or protein with a high activity, and optionally combining these measures. Thus, for example, one or more genes of the group:

the thrABC operon which codes for aspartate kinase, homoserine dehydrogenase, homoserine kinase and threonine synthase (U.S. Pat. No. 4,278,765),

the pyc gene which codes for pyruvate carboxylase (DE-A-19 831 609),

the pps gene which codes for phosphoenol pyruvate synthase (Molecular and General Genetics 231:332 1992)),

the ppc gene which codes for phosphoenol pyruvate carboxylase (Gene 31:279-283 (1984)),

the pntA and pntB genes which code for transhydrogenase (European Journal of Biochemistry 158:647-653 (1986)),

the rhtB gene which imparts homoserine resistance (EP-A-0 994 190),

the mqo gene which codes for malate:quinone oxidoreductase (DE 100 348 33.5),

the rhtC gene which imparts threonine resistance (EP-A-1 013 765), and

the thrE gene of Corynebacterium glutamicum which codes for threonine export (DE 100 264 94.8) and

the gdhA gene which codes for glutamate dehydrogenase (Nucleic Acids Research 11: 5257-5266 (1983); Gene 23: 199-209 (1983)) can be enhanced, in particular over-expressed, at the same time.

It may furthermore be advantageous for the production of L-amino acids, in particular threonine, in addition to the attenuation of the poxB gene, for one or more genes chosen from the group consisting of

the tdh gene which codes for threonine dehydrogenase (Ravnikar and Somerville, Journal of Bacteriology 169, 4716-4721 (1987)),

the mdh gene which codes for malate dehydrogenase (E.C. 1.1.1.37) (Vogel et al., Archives in Microbiology 149, 36-42 (1987)),

the gene product of the open reading frame (orf) yjfA (Accession Number AAC77180 of the National Center for Biotechnology Information (NCBI, Bethesda, Md., USA),

the gene product of the open reading frame (orf) ytfP (Accession Number AAC77179 of the National Center for Biotechnology Information (NCBI, Bethesda, Md., USA) and

the pckA gene which codes for the enzyme phosphoenol pyruvate carboxykinase (Medina et al. (Journal of Bacteriology 172, 7151-7156 (1990)) to be attenuated, in particular eliminated or reduced in expression.

In addition to attenuation of the poxB gene it may furthermore be advantageous for the production of L-amino acids, in particular L-threonine, to eliminate undesirable side reactions (Nakayama: “Breeding of Amino Acid Producing Microorganisms”, in: Overproduction of Microbial Products, Krumphanzl, Sikyta, Vanek (eds.), Academic Press, London, UK, 1982).

The microorganisms produced according to the present invention can be cultured in the batch process (batch culture), the fed batch (feed process) or the repeated fed batch process (feed process). A summary of known culture methods is described in the textbook by Chmiel (Bioprozesstechnik 1. Einführung in die Bioverfahrenstechnik [Bioprocess Technology 1. Introduction to Bioprocess Technology (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981).

Sugars and carbohydrates, such as e.g. glucose, sucrose, lactose, fructose, maltose, molasses, starch and optionally cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substances can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as e.g. magnesium sulfate or iron sulfate, which are necessary for growth. Finally, essential growth substances, such as amino acids and vitamins, can be employed in addition to the abovementioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH. Antifoams, such as e.g. fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually about 25° C. to 45° C., and preferably about 30° C. to 40° C. Culturing is continued until a maximum of L-amino acids or L-threonine has formed. This target is usually reached within about 10 hours to 160 hours.

The analysis of L-amino acids can be carried out by anion exchange chromatography with subsequent ninhydrin derivatization, as described by Spackman et al. (Analytical Chemistry, 30, (1958), 1190), or it can take place by reversed phase HPLC as described by Lindroth et al. (Analytical Chemistry (1979) 51:. 1167-1174).

A pure culture of the Escherichia coli K-12 strain DH5α/pMAK705 was deposited as DSM 13720 on 8th September 2000 at the Deutsche Sammlung für Mikroorganismen und Zellkulturen (DSMZ=German Collection of Microorganisms and Cell Cultures, Braunschweig, Germany) in accordance with the Budapest Treaty. A pure culture of the Escherichia coli K-12 strain MG442ΔpoxB was deposited as DSM 13762 on Oct. 2, 2000 at the Deutsche Sammlung für Mikroorganismen und Zellkulturen (DSMZ=German Collection of Microorganisms and Cell Cultures, Braunschweig, Germany) in accordance with the Budapest Treaty.

The process according to the invention is used for the fermentative preparation of L-amino acids, such as e.g. L-threonine, L-isoleucine, L-valine, L-methionine, L-homoserine and L-lysine, in particular L-threonine.

The present invention is explained will now be explained in more detail in the following embodiment examples which are provided solely for purposes of illustration and are not intended to be limitative.

The isolation of plasmid DNA from Escherichia coli and all techniques of restriction, Klenow and alkaline phosphatase treatment are carried out by the method of Sambrook et al. (Molecular cloning—A laboratory manual (1989) Cold Spring Harbour Laboratory Press). Unless described otherwise, the transformation of Escherichia coli is carried out by the method of Chung et al. (Proceedings of the National Academy of Sciences of the United States of America USA (1989) 86: 2172-2175).

The incubation temperature for the preparation of strains and transformants is 37° C. Temperatures of 30° C. and 44° C. are used in the gene replacement method of Hamilton et. al.

### EXAMPLE 1

### Construction of the Deletion Mutation of the poxB Gene

Parts of the 5′ and 3′ region of the poxB gene are amplified from Escherichia coli K12 using the polymerase chain reaction (PCR) and synthetic oligonucleotides. Starting from the nucleotide sequence of the poxB gene in E. coli K12 MG1655 (SEQ ID No. 1), the following PCR primers are synthesized (MWG Biotech, Ebersberg, Germany):

poxB′5′-1: 5′ - CTGAACGGTCTTAGTGACAG - 3′
poxB′5′-2: 5′ - AGGCCTGGAATAACGCAGCAGTTG - 3′
poxB′3′-1: 5′ - CTGCGTGCATTGCTTCCATTG - 3′
poxB′3′-2: 5′ - GCCAGTTCGATCACTTCATCAC - 3′

The chromosomal E. coli K12 MG1655 DNA employed for the PCR is isolated according to the manufacturers instructions with “Qiagen Genomic-tips 100/G” (QIAGEN, Hilden, Germany). A DNA fragment approx. 500 base pairs (bp) in size from the 5′ region of the poxB gene (called poxB1) and a DNA fragment approx. 750 bp in size from the 3′ region of the poxB gene (called poxB2) can be amplified with the specific primers under standard PCR conditions (Innis et al. (1990) PCR Protocols. A Guide to Methods and Applications, Academic Press) with Taq-DNA polymerase (Gibco-BRL, Eggenstein, Germany). The PCR products are each ligated with the vector pCR2.1TOPO (TOPO TA Cloning Kit, Invitrogen, Groningen, The Netherlands) in accordance with the manufacturers instructions and transformed into the E. coli strain TOP10F′. Selection of plasmid-carrying cells takes place on LB agar, to which 50 μg/ml ampicillin are added. After isolation of the plasmid DNA, the vector pCR2.1TOPOpoxB1is cleaved with the restriction enzymes Ecl136II and XbaI and, after separation in 0.8% agarose gel, the poxB1 fragment is isolated with the aid of the QIAquick Gel Extraction Kit (QIAGEN, Hilden, Germany). After isolation of the plasmid DNA the vector pCR2.1TOPOpoxB2 is cleaved with the enzymes EcoRV and XbaI and ligated with the poxBl fragment isolated. The E. coli strain DH5α is transformed with the ligation batch and plasmid-carrying cells are selected on LB agar, to which 50 μg/ml ampicillin is added. After isolation of the plasmid DNA those plasmids in which the mutagenic DNA sequence shown in SEQ ID No. 3 is cloned are detected by control cleavage with the enzymes HindIII and XbaI. One of the plasmids is called pCR2.1TOPOΔpoxB.

### EXAMPLE 2

### Construction of the Replacement Vector pMAK705ΔpoxB

The poxB allele described in Example 1 is isolated from the vector pCR2.1TOPOΔpoxB after restriction with the enzymes HindIII and XbaI and separation in 0.8% agarose gel, and ligated with the plasmid pMAK705 (Hamilton et al. (1989) Journal of Bacteriology 174, 4617-4622), which has been digested with the enzymes HindIII and XbaI. The ligation batch is transformed in DH5α and plasmid-carrying cells are selected on LB agar, to which 20 μg/ml chloramphenicol is added. Successful cloning is demonstrated after isolation of the plasmid DNA and cleavage with the enzymes HindIII and XbaI. The replacement vector formed, pMAK705ΔpoxB (=pMAK705deltapoxB), is shown in FIG. 1.

### EXAMPLE 3

### Position-specific Mutagenesis of the poxB gene in the E. coli Strain MG442

The L-threonine-producing E. coli strain MG442 is described in the patent specification U.S. Pat. No. 4,278,765 and deposited as CMIM B-1628 at the Russian National Collection for Industrial Microorganisms (VKPM, Moscow, Russia). For replacement of the chromosomal poxB gene with the plasmid-coded deletion construct, MG442 is transformed with the plasmid pMAK705ΔpoxB, The gene replacement is carried out by the selection method described by Hamilton et al. (1989) Journal of Bacteriology 174, 4617-4622) and is verified by standard PCR methods (Innis et al. (1990) PCR Protocols. A Guide to Methods and Applications, Academic Press) with the following oligonucleotide primers:

poxB′5′-1: 5′-CTGAACGGTCTTAGTGACAG-3′

poxB′3′-2: 5′-GCCAGTTCGATCACTTCATCAC -3′

The strain obtained is called MG442ΔpoxB.

### EXAMPLE 4

### Preparation of L-threonine with the Strain MG442ΔpoxB

MG442ΔpoxB is multiplied on minimal medium with the following composition: 3.5 g/l Na₂HPO₄*2H₂O, 1.5 g/l KH₂PO₄, 1 g/l NH₄Cl, 0.1 g/l MgSO₄*7H₂O, 2 g/l glucose, 20 g/l agar. The formation of L-threonine is checked in batch cultures of 10 ml contained in 100 ml conical flasks. For this, 10 ml of preculture medium of the following composition: 2 g/l yeast extract, 10 g/l (NH₄)₂SO₄, 1 g/l KH₂PO₄, 0.5 g/l MgSO₄*7H₂O, 15 g/l CaCO₃, 20 g/l glucose are inoculated and the batch is incubated for 16 hours at 37° C. and 180 rpm on an ESR incubator from Kühner AG (Birsfelden, Switzerland). 250 μl of this preculture are transinoculated into 10 ml of production medium (25 g/l (NH₄)₂SO₄, 2 g/l KH₂PO₄, 1 g/l MgSO₄*7H₂O, 0.03 g/l FeSO₄*7H₂O, 0.018 g/l MnSO₄*1H₂O, 30 g/l CaCO₃, 20 g/l glucose) and the batch is incubated for 48 hours at 37° C. After the incubation the optical density (OD) of the culture suspension is determined with an LP2W photometer from Dr. Lange (Berlin, Germany) at a measurement wavelength of 660 nm.

The concentration of L-threonine formed is then determined in the sterile-filtered culture supernatant with an amino acid analyzer from Eppendorf-BioTronik (Hamburg, Germany) by ion exchange chromatography and post-column reaction with ninhydrin detection.

The result of the experiment is shown in Table 1.

TABLE 1

| | OD | L-Threonine |
|---|---|---|
| Strain | (660 nm) | g/l |
| MG442 | 6.0 | 1.5 |
| MG442ΔpoxB | 4.9 | 2.6 |

### EXAMPLE 5

### Preparation of L-threonine with the Strain MG442ΔpoxB/pMW218gdhA

5.1 Amplification and cloning of the gdhA gene

The glutamate dehydrogenase gene from Escherichia coli K12 is amplified using the polymerase chain reaction (PCR) and synthetic oligonucleotides. Starting from the nucleotide sequence for the gdhA gene in E. coli K12 MG1655 (gene library: Accession No. AE000270 and No. AE000271), PCR primers are synthesized (MWG Biotech, Ebersberg, Germany):

Gdh1: 5′-TGAACACTTCTGGCGGTACG-3′

Gdh2: 5′-CCTCGGCGAAGCTAATATGG-3′

The chromosomal E. coli K12 MG1655 DNA employed for the PCR is isolated according to the manufacturers instructions with “QIAGEN Genomic-tips 100/G” (QIAGEN, Hilden, Germany). A DNA fragment approx. 2150 bp in size, which comprises the gdhA coding region and approx. 350 bp 5′ -flanking and approx. 450 bp 3′ -flanking sequences, can be amplified with the specific primers under standard PCR conditions (Innis et al.: PCR protocols. A guide to methods and applications, 1990, Academic Press) with Pfu-DNA polymerase (Promega Corporation, Madison, USA). The PCR product is cloned in the plasmid pCR2.1TOPO and transformed in the E. coli strain TOP10 (Invitrogen, Leek, The Netherlands, Product Description TOPO TA Cloning Kit, Cat. No. K4500-01). Successful cloning is demonstrated by cleavage of the plasmid pCR2.1TOPOgdhA with the restriction enzymes EcoRI and EcoRV. For this, the plasmid DNA is isolated by means of the “QIAprep Spin Plasmid Kit” (QIAGEN, Hilden, Germany) and, after cleavage, separated in a 0.8% agarose gel.

5.2 Cloning of the gdhA gene in the plasmid vector pMW218

The plasmid pCR2.1TOPOgdhA is cleaved with the enzyme EcoRI, the cleavage batch is separated on 0.8% agarose gel and the gdhA fragment 2.1 kbp in size is isolated with the aid of the “QIAquick Gel Extraction Kit” (QIAGEN, Hilden, Germany). The plasmid pMW218 (Nippon Gene, Toyama, Japan) is cleaved with the enzyme EcoRI and ligated with the gdhA fragment. The E. coli strain DH5α is transformed with the ligation batch and pMW218-carrying cells are selected by plating out on LB agar (Lennox, Virology 1955, 1: 190), to which 20 μg/ml kanamycin are added.

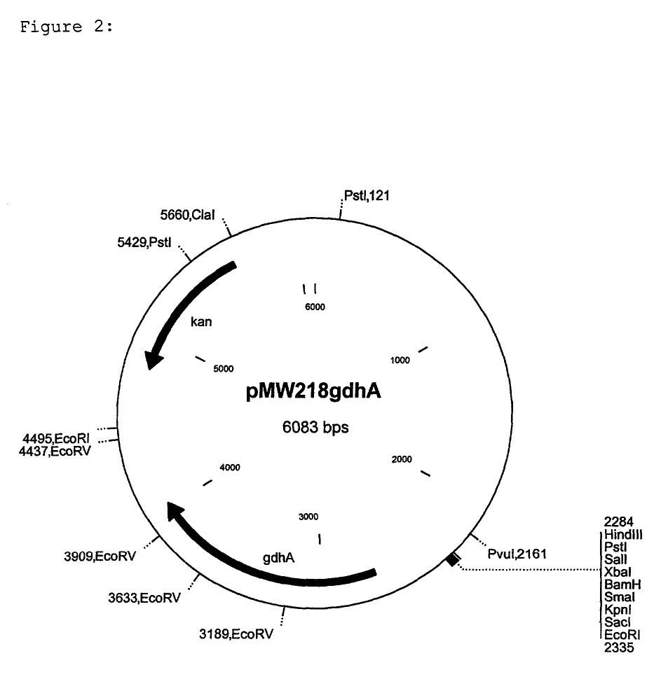

Successful cloning of the gdhA gene can be demonstrated after plasmid DNA isolation and control cleavage with EcoRI and EcoRV. The plasmid is called pMW218gdhA (FIG. 2).

5.3 Preparation of the strain MG442ΔpoxB/pMW218gdhA

The strain MG442ΔpoxB obtained in Example 3 and the strain MG442 are transformed with the plasmid pMW218gdhA and transformants are selected on LB agar, which is supplemented with 20 μg/ml kanamycin. The strains MG442ΔpoxB/pMW218gdhA and MG442/pMW218gdhA are formed in this manner.

5.4 Preparation of L-threonine

The preparation of L-threonine by the strains MG442ΔpoxB/pMW218gdhA and MG442/pMW218gdhA is tested as described in Example 4. The minimal medium and the preculture medium are additionally supplemented with 20 μg/ml kanamycin for these two strains.

### The result of the experiment is summarized in Table 2.

TABLE 2

| | OD | L-Threonine |
|---|---|---|
| Strain | (660 nm) | g/l |
| MG442 | 6.0 | 1.5 |
| MG442ΔpoxB | 4.9 | 2.6 |
| MG442/pMW218gdhA | 5.6 | 2.6 |
| MG442ΔpoxB/pMW218gdhA | 5.5 | 2.9 |

### EXAMPLE 6

### Preparation of L-threonine with the Strain MG442ΔpoxB/pMW219rhtC

6.1 Amplification of the rhtC Gene

The rhtC gene from Escherichia coli K12 is amplified using the polymerase chain reaction (PCR) and synthetic oligonucleotides. Starting from the nucleotide sequence for the rhtC gene in E. coli K12 MG1655 (gene library: Accession No. AE000458, Zakataeva et al. (FEBS Letters 452, 228-232 (1999)), PCR primers are synthesized (MWG Biotech, Ebersberg, Germany):

RhtC1: 5′-CTGTTAGCATCGGCGAGGCA-3′

RhtC2: 5′-GCATGTTGATGGCGATGACG-3′

The chromosomal E. coli K12 MG1655 DNA employed for the PCR is isolated according to the manufacturers instructions with “QIAGEN Genomic-tips 100/G” (QIAGEN, Hilden, Germany). A DNA fragment approx. 800 bp in size can be amplified with the specific primers under standard PCR conditions (Innis et al.: PCR protocols. A guide to methods and applications, 1990, Academic Press) with Pfu-DNA polymerase (Promega Corporation, Madison, USA).

6.2 Cloning of the rhtC Gene in the Plasmid Vector pMW219

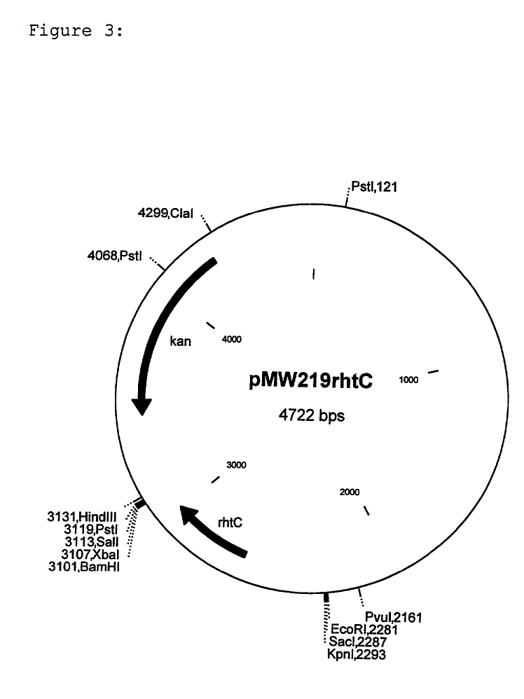

The plasmid pMW219 (Nippon Gene, Toyama, Japan) is cleaved with the enzyme SamI and ligated with the rhtC-PCR fragment. The E. coli strain DH5α is transformed with the ligation batch and pMW219-carrying cells are selected on LB agar, which is supplemented with 20 μg/ml kanamycin. Successful cloning can be demonstrated after plasmid DNA isolation and control cleavage with KpnI, HindIII and NcoI. The plasmid pMW219rhtC is shown in FIG. 3.

6.3 Preparation of the Strain MG442ΔpoxB/pMW219rhtC

The strain MG442ΔpoxB obtained in Example 3 and the strain MG442 are transformed with the plasmid pMW219rhtC and transformants are selected on LB agar, which is supplemented with 20 μg/ml kanamycin. The strains MG442ΔpoxB/pMW219rhtC and MG442/pMW219rhtC are formed in this way.

6.4 Preparation of L-threonine

The preparation of L-threonine by the strains MG442ΔpoxB/pMW219rhtC and MG442/pMW219rhtC is tested as described in Example 4. The minimal medium and the preculture medium are additionally supplemented with 20 μg/ml kanamycin for these two strains.

The result of the experiment is summarized in Table 3.

TABLE 3

| | OD | L-Threonine |
|---|---|---|
| Strain | (660 nm) | g/l |
| MG442 | 6.0 | 1.5 |
| MG442ΔpoxB | 4.9 | 2.6 |
| MG442/pMW219rhtC | 5.2 | 2.9 |
| MG442ΔpoxB/pMW219rhtC | 5.4 | 3.9 |

### EXAMPLE 7

Position-specific Mutagenesis of the poxB Gene in the E. coli strain TOC21R

The L-lysine-producing E. coli strain pDA1/TOC21R is described in the patent application F-A-2511032 and deposited at the Collection Nationale de Culture de Microorganisme (CNCM=National Microorganism Culture Collection, Pasteur Institute, Paris, France) under number I-167. The strain and the plasmid-free host are also described by Dauce-Le Reverend et al. (European Journal of Applied Microbiology and Biotechnology 15:227-231 (1982)) under the name TOCR21/pDA1.

After culture in antibiotic-free LB medium for approximately six generations, a derivative of strain pDA1/TOC21R which no longer contains the plasmid pDA1 is isolated. The strain formed is tetracycline-sensitive and is called TOC21R. For replacement of the chromosomal poxB gene with the plasmid-coded deletion construct, TOC21R is transformed with the plasmid pMAK705ΔpoxB (Example 2). The gene replacement is carried out by the selection method described by Hamilton et al. (1989) Journal of Bacteriology 174, 4617-4622) and is verified by standard PCR methods (Innis et al. (1990) PCR Protocols. A Guide to Methods and Applications, Academic Press) with the following oligonucleotide primers:

poxB′5′-1: 5′-CTGAACGGTCTTAGTGACAG-3′

poxB′3′-2: 5′-GCCAGTTCGATCACTTCATCAC-3′

The strain obtained is called TOC21RΔpoxB.

### EXAMPLE 8

### Preparation of L-lysine with the Strain TOC21RΔpoxB

The formation of L-lysine by the strains TOC21RΔpoxB and TOC21R is checked in batch cultures of 10 ml contained in 100 ml conical flasks. For this, 10 ml of preculture medium of the following composition: 2 g/l yeast extract, 10 g/l (NH₄)₂SO₄, 1 g/l KH₂PO₄, 0.5 g/l MgSO₄*7H₂O, 15 g/l CaCO₃, 20 g/l glucose are inoculated and the batch is incubated for 16 hours at 37° C. and 180 rpm on an ESR incubator from Kühner AG (Birsfelden, Switzerland). 250 μl of this preculture are transinoculated into 10 ml of production medium (25 g/l (NH₄)₂SO₄, 2 g/l KH₂PO₄, 1 g/l MgSO₄*7H₂O, 0.03 g/l FeSO₄*7H₂O, 0.018 g/l MnSO₄*1H₂O, 30 g/l CaCO₃, 20 g/l glucose, 25 mg/l L-isoleucine and 5 mg/l thiamine) and the batch is incubated for 72 hours at 37° C. After the incubation the optical density (OD) of the culture suspension is determined with an LP2W photometer from Dr. Lange (Berlin, Germany) at a measurement wavelength of 660 nm. The concentration of L-lysine formed is then determined in the sterile-filtered culture supernatant with an amino acid analyzer from Eppendorf-BioTronik (Hamburg, Germany) by ion exchange chromatography and post-column reaction with ninhydrin detection.

The result of the experiment is shown in table 4.

TABLE 4

| | OD | L-Lysine |
|---|---|---|
| Strain | (660 nm) | g/l |
| TOC21R | 1.0 | 1.17 |
| TOC21RΔpoxB | 1.0 | 1.29 |

### EXAMPLE 9

### Position-specific Mutagenesis of the poxB Gene in the E. coli Strain B-1288

The L-valine-producing E. coli strain AJ 11502 is described in the patent specification U.S. Pat. No. 4,391,907 and deposited at the National Center for Agricultural Utilization Research (Peoria, Ill., USA) as NRRL B-12288.

After culture in antibiotic-free LB medium for approximately six generations, a plasmid-free derivative of strain AJ 11502 is isolated. The strain formed is ampicillin-sensitive and is called AJ11502kur.

For replacement of the chromosomal poxB gene with the plasmid-coded deletion construct, AJ11502kur is transformed with the plasmid pMAK705ΔpoxB (see Example 2). The gene replacement is carried out by the selection method described by Hamilton et al. (1989) Journal of Bacteriology 174, 4617-4622) and is verified by standard PCR methods (Innis et al. (1990) PCR Protocols. A Guide to Methods and Applications, Academic Press) with the following oligonucleotide primers:

poxB′5′-1: 5′- CTGAACGGTCTTAGTGACAG-3′

poxB′3′-2: 5′-GCCAGTTCGATCACTTCATCAC-3′

The strain obtained is called AJ11502kurΔpoxB. The plasmid described in the patent specification U.S. Pat. No. 4,391,907, which carries the genetic information in respect of valine production, is isolated from strain NRRL B-12288. The strain AJ11502kurΔpoxB is transformed with this plasmid. One of the transformants obtained is called B-12288ΔpoxB.

### EXAMPLE 10

### Preparation of L-valine with the Strain B-12288ΔpoxB

The formation of L-valine by the strains B-12288ΔpoxB and NRRL B-12288 is checked in batch cultures of 10 ml contained in 100 ml conical flasks. For this, 10 ml of preculture medium of the following composition: 2 g/l yeast extract, 10 g/l (NH₄)₂SO₄, 1 g/1 KH₂PO₄, 0.5 g/l MgSO₄*7H₂O, 15 g/l CaCO₃, 20 g/l glucose and 50 mg/l ampicillin are inoculated and the batch is incubated for 16 hours at 37° C. and 180 rpm on an ESR incubator from Kühner AG (Birsfelden, Switzerland). 250 μl of this preculture are transinoculated into 10 ml of production medium (25 g/l (NH₄)₂SO₄, 2 g/l KH₂PO₄, 1 g/l MgSO₄*7H₂O, 0.03 g/l FeSO₄*7H₂O, 0.018 g/l MnSO₄*1H₂O, 30 g/l CaCO₃, 20 g/l glucose, 5 mg/l thiamine and 50 mg/l ampicillin) and the batch is incubated for 72 hours at 37° C. After the incubation the optical density (OD) of the culture suspension is determined with an LP2W photometer from Dr. Lange (Berlin, Germany) at a measurement wavelength of 660 nm.

The concentration of L-valine formed is then determined in the sterile-filtered culture supernatant with an amino acid analyzer from Eppendorf-BioTronik (Hamburg, Germany) by ion exchange chromatography and post-column reaction with ninhydrin detection.

The result of the experiment is shown in table 5.

TABLE 5

| | CD | L-Valine |
|---|---|---|
| Strain | (660 nm) | g/l |
| NRRL B-12288 | 5.7 | 0.95 |
| B-12288ΔpoxB | 5.6 | 1.05 |

### BRIEF DESCRIPTION OF THE FIGURES

FIG. 1: pMAK705ΔpoxB (=pMAK705deltapoxB)

FIG. 2: pMW218gdhA

FIG. 3: pMW219rhtC

The length data are to be understood as approx. data. The abbreviations and designations used have the following meaning:

cat: chloramphenicol resistance gene

rep-ts: temperature-sensitive replication region of the plasmid pSC101

poxB1: part of the 5′ region of the poxB gene

poxB2: part of the 3′ region of the poxB gene

kan: kanamycin resistance gene

gdhA: glutamate dehdyrogensase gene

rhtC: gene imparting threonine resistance

The abbreviations for the restriction enzymes have the following meaning

BamHI: restriction endonuclease from Bacillus amyloliquefaciens

BglII: restriction endonuclease from Bacillus globigii

ClaI: restriction endonuclease from Caryphanon latum

Ecl136II restriction endonuclease from Enterobacter cloacae RFL136 (=Ecl136)

EcoRI: restriction endonuclease from Escherichia coli

EcoRV: restriction endonuclease from Escherichia coli

HindIII: restriction endonuclease from Haemophilus influenzae

KpnI: restriction endonuclease from Klebsiella pneumoniae

PstI: restriction endonuclease from Providencia stuartii

PvuI: restriction endonuclease from Proteus vulgaris

SacI: restriction endonuclease from Streptomyces achromogenes

SalI: restriction endonuclease from Streptomyces albus

SmaI: restriction endonuclease from Serratia marcescens

XbaI: restriction endonuclease from Xanthomonas badrii

XhoI: restriction endonuclease from Xanthomonas holcicola

Having described the present invention, it will be apaprent to one of ordinary skill in the art that many changes and modifications may be made to the above-described embodiments without departing from the spirit and scope of the present invention.

What is claimed is:

1. A process for fermentatively preparing an L-amino acid, which comprises the steps of:
a) fermenting microorganisms of the Enterobacteriaceae family which produce an L-amino acid and in which at least poxB gene or nucleotide sequences which code therefor are attenuated or eliminated;
b) concentrating the L-amino acid in the medium or in the cells of the bacteria; and
c) isolating the L-amino acid.

2. The process of claim 1, wherein said L-amino acid prepared is L-threonine, L-valine, L-lysine, L-isoleucine, L-methionine, or L-homoserine.

3. The process of claim 1, wherein said microorganisms have additional genes of the biosynthesis pathway of the L-amino acid additionally enhanced.

4. The process of claim 1, wherein said microorganisms have metabolic pathways which reduce formation of the L-amino acid which are at least partly eliminated.

5. The process of claim 1, wherein expression of the polynucleotide(s) which code(s) for the poxB gene is attenuated or eliminated.

6. The process of claim 1, wherein regulatory or catalytic properties or both of the polypeptide for which the polynucleotide poxB codes are reduced.

7. The process of claim 1, which comprises fermenting, for the preparation of the L-amino acid, microorganisms of the Enterobacteriaceae family in which one or more genes selected from the group consisting of:
1) the thrABC operon which codes for aspartate kinase, homoserine dehydrogenase, homoserine kinase and threonine synthase,
2) the pyc gene which codes for pyruvate carboxylase,
3) the pps gene which codes for phosphoenol pyruvate synthase,
4) the ppc gene which codes for phosphoenol pyruvate carboxylase,
5) the pntA and pntB genes which code for transhydrogenase,
6) the rhtB gene which imparts homoserine resistance,
7) the mqo gene which codes for malate:quinone oxidoreductase,
8) the rhtC gene which imparts threonine resistance,
9) the thrE gene which codes for threonine export, and
10) the gdhA gene which codes for glutamate dehydrogenase, is or are enhanced at the same time.

8. The process of claim 7, wherein said one or more genes are over-expressed.

9. The process of claim 1, which comprises fermenting, for the preparation of L-amino acids, microorganisms of the Enterobacteriaceae family in which one or more genes chosen from the group consisting of:
1) the tdh gene which codes for threonine dehydrogenase,
2) the mdh gene which codes for malate dehydrogenase,
3) the gene product of the open reading frame (orf) yjfA,
4) the gene product of the open reading frame (orf) ytfP, and
5) the pckA gene which codes for the enzyme phosphoenol pyruvate carboxykinase, is or are attenuated at the same time.

10. The process of claim 9, wherein said one or more genes are eliminated or reduced in expression.

11. The process of claim 2, wherein said L-amino acid is selected from the group consisting of L-threonine, L-valine and L-lysine.

12. The process of claim 1, which comprises employing, for the preparation of L-threonine, strain MG442ΔpoxB transformed with plasmid pMW218gdhA, shown in FIG. 2.

13. The process of claim 1, which comprises employing, for preparation of L-threonine, strain MG442ΔpoxB transformed with plasmid pMW219rhtC, shown in FIG. 3.

14. The process of claim 1, which comprises employing, for preparation of L-lysine, strain TOC21RΔpoxB.

15. The process of claim 1, which comprises employing, for preparation of L-valine, strain B-12288ΔpoxB.

16. A microorganism of the Enterobacteriaceae family which produces an L-amino acid, in which poxB gene or nucleotide sequences coding therefor are attenuated, or eliminated, and which have resistance to α-amino-β-hydroxyvaleric acid and optionally a compensatable partial need for L-isoleucine.

17. Escherichia coli K-12 strain MG442ΔpoxB deposited at the Deutsche Sammlung für Mikroorganismen und Zellkulturen (DSMZ=German Collection of Microorganisms and Cell Cultures, Braunschweig, Germany) under no. DSM 13762.

18. Plasmid pMAK705ΔpoxB, which comprises parts of the 5′ and of the 3′ region of poxB gene, corresponding to SEQ ID No. 3, shown in FIG. 1.

19. Plasmid pMW218gdhA shown in FIG. 2.

20. Plasmid pMW219rhtC shown in FIG. 3.

21. An isolated polynucleotide from microorganisms of the Enterobacteriaceae family, containing a polynucleotide sequence which codes for the 5′ and 3′ region of poxB gene, shown in SEQ ID No. 4, which is capable of being used as a constituent of plasmids for position-specific mutagenesis of poxB gene.

22. A strain of the Enterobacteriaceae family which produces L-threonine and contains a mutation in the poxB gene, corresponding to SEQ ID No. 4.


SEQUENCE LISTING (12 sequences)

SEQ ID NO: 1 - DNA, 1719 bp, Escherichia coli
  CDS (1)..(1716)
atgaaacaaacggttgcagcttatatcgccaaaacactcgaatcggcaggggtgaaacgcatctggggagtcacaggcgactctctgaacggtcttagtgacagtcttaatcgcatgggcaccatcgagtggatgtccacccgccacgaagaagtggcggcctttgccgctggcgctgaagcacaacttagcggagaactggcggtctgcgccggatcgtgcggccccggcaacctgcacttaatcaacggcctgttcgattgccaccgcaatcacgttccggtactggcgattgccgctcatattccctccagcgaaattggcagcggctatttccaggaaacccacccacaagagctattccgcgaatgtagtcactattgcgagctggtttccagcccggagcagatcccacaagtactggcgattgccatgcgcaaagcggtgcttaaccgtggcgtttcggttgtcgtgttaccaggcgacgtggcgttaaaacctgcgccagaaggggcaaccatgcactggtatcatgcgccacaaccagtcgtgacgccggaagaagaagagttacgcaaactggcgcaactgctgcgttattccagcaatatcgccctgatgtgtggcagcggctgcgcgggggcgcataaagagttagttgagtttgccgggaaaattaaagcgcctattgttcatgccctgcgcggtaaagaacatgtcgaatacgataatccgtatgatgttggaatgaccgggttaatcggcttctcgtcaggtttccataccatgatgaacgccgacacgttagtgctactcggcacgcaatttccctaccgcgccttctacccgaccgatgccaaaatcattcagattgatatcaacccagccagcatcggcgctcacagcaaggtggatatggcactggtcggcgatatcaagtcgactctgcgtgcattgcttccattggtggaagaaaaagccgatcgcaagtttctggataaagcgctggaagattaccgcgacgcccgcaaagggctggacgatttagctaaaccgagcgagaaagccattcacccgcaatatctggcgcagcaaattagtcattttgccgccgatgacgctattttcacctgtgacgttggtacgccaacggtgtgggcggcacgttatctaaaaatgaacggcaagcgtcgcctgttaggttcgtttaaccacggttcgatggctaacgccatgccgcaggcgctgggtgcgcaggcgacagagccagaacgtcaggtggtcgccatgtgcggcgatggcggttttagcatgttgatgggcgatttcctctcagtagtgcagatgaaactgccagtgaaaattgtcgtctttaacaacagcgtgctgggctttgtggcgatggagatgaaagctggtggctatttgactgacggcaccgaactacacgacacaaactttgcccgcattgccgaagcgtgcggcattacgggtatccgtgtagaaaaagcgtctgaagttgatgaagccctgcaacgcgccttctccatcgacggtccggtgttggtggatgtggtggtcgccaaagaagagttagccattccaccgcagatcaaactcgaacaggccaaaggtttcagcctgtatatgctgcgcgcaatcatcagcggacgcggtgatgaagtgatcgaactggcgaaaacaaactggctaaggtaa

SEQ ID NO: 2 - protein, 572 aa, Escherichia coli
MKQTVAAYIAKTLESAGVKRIWGVTGDSLNGLSDSLNRMGTIEWMSTRHEEVAAFAAGAEAQLSGELAVCAGSCGPGNLHLINGLFDCHRNHVPVLAIAAHIPSSEIGSGYFQETHPQELFRECSHYCELVSSPEQIPQVLAIAMRKAVLNRGVSVVVLPGDVALKPAPEGATMHWYHAPQPVVTPEEEELRKLAQLLRYSSNIALMCGSGCAGAHKELVEFAGKIKAPIVHALRGKEHVEYDNPYDVGMTGLIGFSSGFHTMMNADTLVLLGTQFPYRAFYPTDAKIIQIDINPASIGAHSKVDMALVGDIKSTLRALLPLVEEKADRKFLDKALEDYRDARKGLDDLAKPSEKAIHPQYLAQQISHFAADDAIFTCDVGTPTVWAARYLKMNGKRRLLGSFNHGSMANAMPQALGAQATEPERQVVAMCGDGGFSMLMGDFLSVVQMKLPVKIVVFNNSVLGFVAMEMKAGGYLTDGTELHDTNFARIAEACGITGIRVEKASEVDEALQRAFSIDGPVLVDVVVAKEELAIPPQIKLEQAKGFSLYMLRAIISGRGDEVIELAKTNWLR

SEQ ID NO: 3 - DNA, 1454 bp, Escherichia coli
  misc_feature (1)..(1454): mutagenic DNA
ctagatgcatgctcgagcggccgccagtgtgatggatatctgcagaattcgcccttctgaacggtcttagtgacagtcttaatcgcatgggcaccatcgagtggatgtccacccgccacgaagaagtggcggcctttgccgctggcgctgaagcacaacttagcggagaactggcggtctgcgccggatcgtgcggccccggcaacctgcacttaatcaacggcctgttcgattgccaccgcaatcacgttccggtactggcgattgccgctcatattccctccagcgaaattggcagcggctatttccaggaaacccacccacaagagctattccgcgaatgtagtcactattgcgagctggtttccagcccggagcagatcccacaagtactggcgattgccatgcgcaaagcggtgcttaaccgtggcgtttcggttgtcgtgttaccaggcgacgtggcgttaaaacctgcgccagaaggggcaaccatgcactggtatcatgcgccacaaccagtcgtgacgccggaagaagaagagttacgcaaactggcgcaactgctgcgttattccaggcctaagggcgaattccagcacactggcggccgttactagtggatccgagatctgcagaattcgcccttctgcgtgcattgcttccattggtggaagaaaaagccgatcgcaagtttctggataaagcgctggaagattaccgcgacgcccgcaaagggctggacgatttagctaaaccgagcgagaaagccattcacccgcaatatctggcgcagcaaattagtcattttgccgccgatgacgctattttcacctgtgacgttggtacgccaacggtgtgggcggcacgttatctaaaaatgaacggcaagcgtcgcctgttaggttcgtttaaccacggttcgatggctaacgccatgccgcaggcgctgggtgcgcaggcgacagagccagaacgtcaggtggtcgccatgtgcggcgatggcggttttagcatgttgatgggcgatttcctctcagtagtgcagatgaaactgccagtgaaaattgtcgtctttaacaacagcgtgctgggctttgtggcgatggagatgaaagctggtggctatttgactgacggcaccgaactacacgacacaaactttgcccgcattgccgaagcgtgcggcattacgggtatccgtgtagaaaaagcgtctgaagttgatgaagccctgcaacgcgccttctccatcgacggtccggtgttggtggatgtggtggtcgccaaagaagagttagccattccaccgcagatcaaactcgaacaggccaaaggtttcagcctgtatatgctgcgcgcaatcatcagcggacgcggtgatgaagtgatcgaactggcaagggcgaattccagcacactggcggccgttactagtggatccgagctcggtacca

SEQ ID NO: 4 - DNA, 720 bp, Escherichia coli
  misc_feature (1)..(3): start codon of the delta poxB allele
atgaaacaaacggttgcagcttatatcgccaaaacactcgaatcggcaggggtgaaacgcatctggggagtcacaggcgactctctgaacggtcttagtgacagtcttaatcgcatgggcaccatcgagtggatgtccacccgccacgaagaagtggcggcctttgccgctggcgctgaagcacaacttagcggagaactggcggtctgcgccggatcgtgcggccccggcaacctgcacttaatcaacggcctgttcgattgccaccgcaatcacgttccggtactggcgattgccgctcatattccctccagcgaaattggcagcggctatttccaggaaacccacccacaagagctattccgcgaatgtagtcactattgcgagctggtttccagcccggagcagatcccacaagtactggcgattgccatgcgcaaagcggtgcttaaccgtggcgtttcggttgtcgtgttaccaggcgacgtggcgttaaaacctgcgccagaaggggcaaccatgcactggtatcatgcgccacaaccagtcgtgacgccggaagaagaagagttacgcaaactggcgcaactgctgcgttattccaggcctaagggcgaattccagcacactggcggccgttactagtggatccgagatctgcagaattcgcccttctgcgtgcattgcttccattggtggaagaaaaagccgatcgcaagt

SEQ ID NO: 5 - DNA, 20 bp, Artificial Sequence
  Synthetic DNA
ctgaacggtcttagtgacag

SEQ ID NO: 6 - DNA, 24 bp, Artificial Sequence
  Synthetic DNA
aggcctggaataacgcagcagttg

SEQ ID NO: 7 - DNA, 21 bp, Artificial Sequence
  Synthetic DNA
ctgcgtgcattgcttccattg

SEQ ID NO: 8 - DNA, 22 bp, Artificial Sequence
  Synthetic DNA
gccagttcgatcacttcatcac

SEQ ID NO: 9 - DNA, 20 bp, Artificial Sequence
  Synthetic DNA
tgaacacttctggcggtacg

SEQ ID NO: 10 - DNA, 20 bp, Artificial Sequence
  Synthetic DNA
cctcggcgaagctaatatgg

SEQ ID NO: 11 - DNA, 20 bp, Artificial Sequence
  Synthetic DNA
ctgttagcatcggcgaggca

SEQ ID NO: 12 - DNA, 20 bp, Artificial Sequence
  Synthetic DNA
gcatgttgatggcgatgacg